(19) United States
(12) Patent Application Publication
(10) Pub. No.: US 2025/0331720 A1
(43) Pub. Date: Oct. 30, 2025

(54) Remote Health Monitoring System

(71) Applicant: LifeQ B.V., Amsterdam (NL)

(72) Inventors: Laurence Richard Olivier, Alpharetta, GA (US); Franco du Preez, Cumming, GA (US); Etienne Terblanche, Alpharetta, GA (US)

(21) Appl. No.: 18/709,958

(22) PCT Filed: Nov. 17, 2022
(86) PCT No.: PCT/US2022/080048

§ 371 Date: May 14, 2024

Related U.S. Application Data
(60) Provisional application No. 63/280,389, filed on Nov. 17, 2021.

(51) Int. Cl. A61B 5/00; G16H 10/60

(52) U.S. Cl. CPC A61B 5/0022; A61B 5/7275; G16H 10/60

(57) ABSTRACT

A health monitoring system is provided including at least one non-invasive wearable device capable of collecting and storing data, and external monitoring devices that displays and analyses the data for accurate monitoring and anomaly detection. The wearable devices are configured to collect low-latency PPG-derived bio-signals and can utilize multiple devices for accuracy and continuity. The system may further include a dashboard that analyzes the information as well as displaying basic information such as number of beds (in-use, available), staff-to-patient ratio, etc. The data can be collected and accessed remotely and may be utilized before, during, and/or after a patient is dispatched from a clinical setting.

### CROSS-REFERENCE TO RELATED APPLICATIONS

The present invention claims priority to U.S. Provisional Application No. 63/280,389, filed on Nov. 17, 2021, the entirety of which is incorporated by reference.

### FIELD OF INVENTION

The present invention is related to the field of non-invasive digital health monitoring, physiological signal processing, and computation of biological data. In particular, the present invention relates to systems and methods to provide real-time access to physiological data.

### BACKGROUND OF THE INVENTION

The present state of the art fails to have an accurate, efficient, and accessible method for remote monitoring of an individual's health. The problem with the current state of the art is that generally, there is not an effective way of knowing someone's physiology while they are not being monitored by a human or by invasive and expensive devices. Further, the current methods are not cost-effective, scalable, or an accurate way to monitor a person continuously and in near real-time; they are focused on monitoring a person who is already determined to be sick.

Apart from lifestyle fitness trackers and mobile applications, there is very little physiological data collected from healthy people prior to hospitalization or an alert of a health concern. This type of information can be instrumental in prevention and recovery in health and wellbeing. For example, a missing aspect in the current state of the art is when someone has a heart attack or a stroke, there are usually prior physiological indicators that were not monitored or observed that would have been useful for a doctor to know. Another problem with the current state of the art is that most elderly care systems utilize an SOS button to alert caregivers and healthcare providers that there is an emergency. These can cause delay, false positives, and a need for the user to be conscious/aware of their situation. Further, it is not particularly helpful to collect data from a sick individual without any data of the individual when healthy to which to compare.

When someone goes to a primary care physician (PCP) for their yearly checkup, the PCP is only equipped with observable data during the time that the patient is in the doctor's office. Further, this type of patient monitoring is rare and sporadic, like a series of snapshots. There is no method for constantly monitoring and collecting information about an individual, let alone, a large number of people simultaneously and continuously. There is also no method of collecting real time data (such as temperature sensing, blood pressure, Sp02, and heart rate reading) before a patient comes to the doctor for a visit. As the use of telehealth increases, so too must the information available to doctor's remotely improve in breadth and accuracy.

In terms of continuity, current systems 20, as shown in FIG. 1, require that a backend of an application “requests” information from monitoring equipment (for example a wearable device). All the control for the interaction within the system happens at the core/cloud backend. The time it takes to use an edge computing device 201 to wake up the wearable application 205 and upload the data to the core 203 causes a delay and doesn't allow for real-time data collection and alert or alarm generation.

Another major pain point in the current state of the art relating to remote health monitoring is patients at a health facility, whether that be in a hospital, rehab center, elderly care facility, or anything of the like. Any place where there are nurses or doctors would benefit from being able to track the health of the patients. For example, a hospital would benefit from being able to track all of its patients, including those currently in the hospital ward, out-patients, patients in recovery, and patients that are at home before or in-between treatments. When an individual is undergoing treatment or recovery procedures, certain information about that individual is important for the various members of the hospital staff to acquire. At hospitals, nurses and hospital staff are limited into the data they can collect from patients and cannot consistently monitor a patient at a more ideal frequency. Additionally, current monitoring devices employed by hospitals are have additional shortcomings. For example, patches that monitor a patient's sleep are invasive, expensive, and cannot be worn continuously without replacement. Further, in most hospital wards there are not enough resources to acquire a sleep analysis of every patient every day. And the information available from these invasive devices is limited (e.g., cannot continuously track sleep of all patients in the ward).

Some of these pain points were exacerbated during the COVID-19 crisis. There was no way to track and monitor the health, location, and recovery of both healthy and sick individuals, regardless of location. There was no effective way to accurately contact-trace or receive health data that would warn of potential exposure well in advance of a COVID test. Even within the hospitals, doctors and nurses did not have the necessary tools to allow them to view and compare the clinical status between a group of patients and doctors/nurses being required to complete a ward round in order to receive updates on patient status. Hospitals were understaffed during these times. But even more importantly, the staff was busy monitoring everyone and were unable to immediately, consistently, and accurately update their triage list.

While the actual data needed to solve these issues is available, the data was not being transformed or collected in a way that would be most beneficial to clinicians. In current standard practice, a is person dedicated to physically visiting all the wards and reporting back to the matron to determine whether patients may be moved around. Even if the data was collected, access is limited; a doctor at home could not access current physiological data of every given patient at any time point. And access to data is not enough: data in an unorganized manner is further unhelpful in triaging and prioritizing patients.

Apart from everyday users and patients, other stakeholders dealing with the pain points of the current state of the art are hospitals themselves. Hospitals don't have internet of things (IoT) tools to monitor the activity of nurses at the wards. This is problematic because the nursing staff is one of the most expensive expenditures of the hospital and there is no online optimization tool to monitor their staff.

Another important, missing element in the current state of the art, is that not only does the current process lack an efficient mechanism to monitor nurses and staff in a hospital setting, but it does not contribute to resource allocation and resource prioritization. Further, there is a need to expand hospital resource management beyond the confines of the staff and resources available in the hospital.

Overall, there is a lack of ability to monitor all people inside healthcare facilities and even healthy patient monitoring systems are inefficient, costly, and/or invasive. In the current state of the art, there are RFID systems that monitor patients and tracking hospital staff and nurses. However, due to the impracticality and expense of the systems, they have not been effective.

Therefore, there is a need to address the shortcomings addressed above. There is a need to find a solution that allows for continuous monitoring of physiological data of patients and a method to organize that data in a useful manner. There is a need for the data to be remotely and easily accessible so that the timeliness of the data does not expire and to provide for more accurate, current readings of a person's health data. Further, there is a need to monitor users not only when they are sick and admitted into a hospital ward.

### SUMMARY OF THE INVENTION

It is to be understood that this summary is not an extensive overview of the disclosure. This summary is exemplary and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

The present invention is directed at a remote health monitoring system (RHMS) which aims to provide individual users, clinicians, caregivers, and the like, the ability to monitor healthy individuals and sick patients smartly, continuously, cheaply, and in near-real time. The RHMS also enables the monitoring and optimization of patient tracking and the monitoring and allocation of clinical staff and other clinical resources. This enhanced system expands current state of the art remote patient monitoring (RPM) systems beyond just the patient, as it manages the optimal deployment of scares clinical staff and resources. The RHMS utilizes non-invasive instruments (e.g., wearable devices) which stream photoplethysmography (PPG) derived vital signs, as well as other data generated signals, to a patient dashboard (Dashboard) with low-latency, which collects and analyzes the information collected from the wearable devices, and allows users to interact with the data in a meaningful way.

The aim of the RHMS is to provide improved health care through equipping clinicians with the ability to access near real-time remote patient updates and high-resolution patient histories while building a user health history and profile for later use. In an aspect, the RHMS can be configured to generate a dynamic patient stability score that could be used for patient ranking, enabling clinicians to save time to focus on their most critical patients both at the hospital/doctor's office and away. In addition, the RHMS can provide clinicians and other healthcare providers (collectively “monitors”) with a remote user interface (referred to as a Dashboard) that provides such data, health history, and dynamic patient stability scores. Additionally, the remote nature of the Dashboard allows monitors to review user data without the patient being physically present (i.e. patient is remote, doctor is remote, or both). The RHMS, via the Dashboard, also allows the monitors to view the user's information while they are away from the hospital/office. In addition to vital signs, the RHMS can store data derived from previously collected wearable data, which includes information on health relevant contexts such as behavior and physiology, for example, details on sleep stages, efficiency, or amount of sleep. Additional data from additional devices or input by the user and/or physician (such as demographic, age, medical records, and weight from a connected scale) may also be added and stored by the RHMS. It is known that the hospital environment is generally not conducive to optimal sleep, which is an important physiological process for patient recovery and measuring sleep is a crucial step toward solving this problem.

In an aspect, the RHMS is as a less invasive solution to patient monitoring within a hospital ward, providing a solution that can be used over a long period of time and is modular in nature, through the use of wearable devices, as opposed to discardable/consumable patches or other discontinuous solutions. The RHMS provides a less expensive monitoring system that can be sterilized and reused on new users, as well as continuously on current users. Further, the RHMS can provide 24/7 monitoring, as well as directly accessible data, which can help to dispatch on a quicker timescale to patients (15 minute or shorter response time). The ability to swap out wearables and not break continuity allows for continuous monitoring with no gaps in the intake of information.

Further, the RHMS can generate a dynamic risk score associated with a patient, according to an aspect of the present invention. A risk score that is generated upon admission can be initially helpful. However, a patient's health can change after admission. The RHMS can provide a dynamic risk score that can be updated continuously (e.g., every fifteen minutes) to reflect the patient's current health state, which helps properly triage patients and can be used to inform changes in nursing/visit frequency for each patient. The RHMS is a significantly more dynamic and cost-effective approach to current methods of patient monitoring. The RHMS has many additional benefits over conventional, current patient monitoring procedures. In the hospital context, for example, such the RHMS allows hospitals: to facilitate early patient discharge while continuing to monitor treatment outcomes; coping with high patient volumes by monitoring low-risk patients remotely, and admitting high-risk patients for inpatient care; allowing for a bird's eye view of all patients (e.g. when a patient needs to go to the ICU v. when a patient can be at home); and facilitating effective resource allocation (e.g. hospital beds, critical care resources, targeted drug therapies, nursing staff/hours). The RHMS assists nurses in making informed decisions regarding staff resourcing, allocation of nursing staff, improved traceability of ward capacity, and high-resolution and up-to-date information on which to base ward-patient allocation.

The dashboard of the RHMS allows for consolidation in ward management metrics in one place to monitor performance, can track doctor-patient allocation to divide workload more evenly, and consolidates/creates high-level summaries of patient information which allows ward managers to understand what is happening in the ward without interrupting the nurses. Further, the dashboard reduces workload since physicians may check on patients without phoning into the ward to request a report.

While a user is out of the hospital or before they are under a doctor's care, clinicians, through the RHMS, through the use of the non-invasive instruments (e.g., wearable devices) continuous communication, are able to track outcomes of a user's progress between treatments and their physiology under ‘free-living’ conditions. This also allows the users to possibly receive medical attention from a more comfortable/cheaper setting. It also may comfort the patient to know that the clinician has an objective record of physiology to consult as opposed to subjective discussions of symptoms.

Other stakeholders include the patients' loved ones who will be able to receive key updates on patient status and a reassurance that the patient is being actively monitored frequently. Additionally, health insurance agencies will reduce their hospitalization costs due to early patient discharge. Another possible stakeholder in pharmaceutical companies and laboratories that want to conduct studies and trials for new drugs, treatments, and/or to survey a group.

In an aspect, the invention is directed towards a system for non-invasive health monitoring that includes a non-invasive instrument and a core, both of which include processors and are configured to communicate wirelessly with one another and other devices. The non-invasive instrument is also configured to acquire at least vital sign data of a user using the non-invasive instrument. The non-invasive instrument and/or the core are configured to process the vital signal data to generate biological metrics, process the biological metrics to reflect a real-time user health state reflecting physiological data and behavioral data of the user, and detect deviations in the physiological data and the behavioral data of the user from the real-time user health-state. In some instances, the non-invasive instrument is a wearable device that includes at least one photoplethysmography (PPG) sensor to obtain the at least vital sign data of the user.

In an aspect, the real-time user health state can be reflected by generating a patient stability score, wherein the patient stability score is calculated by awarding points based on the physiological data of the user in relation to clinical thresholds. This patient stability score can be utilized for low-latency patient triaging. In some aspects, the system is configured to generate a baseline physiological state of the user from the biological metrics collected over a period of time, wherein the detection of deviations takes into consideration the baseline physiological state of the user.

In some aspects, the health monitoring system can include additional devices that capture medically relevant data of the user and communicate wirelessly with the non-invasive instrument and/or the core, and wherein the core or the non-invasive instrument is configured to process the medically relevant data from the additional device(s) with the biological metrics to reflect the real-time health state of the user.

The remote health monitoring system can also include a remote user interface configured to be in wireless communication with the core or the non-invasive instrument. The remote user interface can also access the biological metrics, the real-time user health state, the physiological data, the behavioral data, and all other information related to the health of the user. Further, the core and the non-invasive instrument can be configured to send alert messages to the remote user interface based upon the physiological data of the user in relation to the clinical thresholds. In addition, the remote health monitoring system can have access to electronic heath records of the user, and can then use the electronic health records, the patient stability score, and the detected deviations in combination to assess a physiological status of the user.

The system can utilize a plurality of non-invasive wearable devices. These wearable devices can be configured to communicate with each other to determine which one of them should collect data when more than one is in use. In some instances, the devices can be configured to simultaneously collect data between two or more of the devices during a transition period (e.g., switching from one with low power remaining to another at full charge) to maintain data continuity. In other aspects, the devices can communicate with each other to determine which should be using battery power and which should be preserving battery power at a given point. Further, the devices can be configured to share data with each other to ensure continuity in battery life as well as data collection.

In one aspect, the system can be configured to continuously monitor the user to collect general health lifestyle information to generate healthy physiological state and health status of the user. The collected general health lifestyle information of the user can be collected during a healthy period. The general lifestyle information can be used to identify a cause of a new medical condition arising during a later period for the user, and/or be used to predict disease course in the instance of a new disease arising. An intervention plan can be generated based on the prognosis derived from the general health lifestyle information of the user plus measured behavior of the user.

In an aspect, the core of the system can be configured to send the real-time user health state and the detected deviations of the user to one or more authorized persons, wherein the authorized person includes a family member, a caretaker, or a medical health professional. In other aspects, the real-time user health state and the detected deviations of the user may be sent to one or more authorized group, wherein the authorized group is a pharmaceutical company, a health insurance company, or a medical company.

In an aspect, the non-invasive instrument of the system can be configured to detect proximity data of at least one or more non-invasive instruments using near field communication. The proximity data can be utilized for prediction and allocation of clinical staff and resources and/or to determine contact amount between users of the one or more non-invasive instruments and clinical staff. In an aspect, the non-invasive instrument can be configured to detect proximity of at least one or more medical devices.

These and other aspects of the invention are discussed in detail below.

### DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a health monitoring system according to the prior art.

FIG. 2 is an overview of a remote health monitoring system according to an aspect of the present invention.

FIG. 3 is a schematic representation of the remote health monitoring system of FIG. 2 according to an aspect of the present invention.

FIG. 4 is an overview of a remote health monitoring system according to an aspect of the present invention.

FIG. 5 is an overview of a remote health monitoring system according to an aspect of the present invention.

### DETAILED DESCRIPTION OF THE INVENTION

The present disclosure can be understood more readily by reference to the following detailed description, examples, drawings, and claims, and their previous and following description. However, before the present compositions, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

### Definitions

Clinician—Any health professional who works one-on-one with patients, diagnosing or treating illness (e.g., doctor, nurse, psychologist).

Doctor/physician—A person qualified to practice medicine (e.g., doctor, medical practitioner).

Caregiver—someone who regularly looks after a sick/elderly/disabled person.

Medical—Relating to medical science.

Clinical—of, relating to, or conducted in, as if in a clinic.

Monitor—clinicians, study monitor, coach, trainer, healthcare specialist, computer, trainer. Someone/something with access to the user's data.

User—in-patient, out-patient, healthy user, athletes, elderly person associated with a non-invasive instrument (e.g., wearing a wearable device 105).

Core—the server (including cloud services) where data streams and biometrics, collected by non-invasive instruments (e.g., wearable devices) associated with users are aggregated and processed into health insights.

The following description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples and are not limited to those set forth herein and may be changed as will be apparent to one of ordinary skill in the art. Description of functions and constructions that are well known to one of ordinary skill in the art may be omitted for increased clarity and conciseness.

The aim of the remote health monitoring system 10 (RHMS) is to provide a method for monitoring health of patients more efficiently both remotely and in clinical settings. The enhanced RHMS 10 utilizes wearable device(s) 105, worn by patients, which streams signal data that corresponds to the wearer's physiological parameters. The wearable device(s) may, for example, be wrist-worn wearable devices, blood pressure cuffs, hospital equipment, et. The data streams collected by the device(s) 105 can include, but are not limited to, photoplethysmography (PPG) signal. In an aspect, the wearable devices 105 utilized by the RHMS 10 can be any wearable device 105 that is configured to capture and derive physiological data from a user, similar to those disclosed in U.S. Pat. No. 9,820,656 (issued Nov. 21, 2017); U.S. Pat. No. 11,129,568 (issued Sep. 28, 2021); and U.S. Pat. No. 11,291,392 (issued Apr. 5, 2022), the entirety of which are incorporated by reference herein.

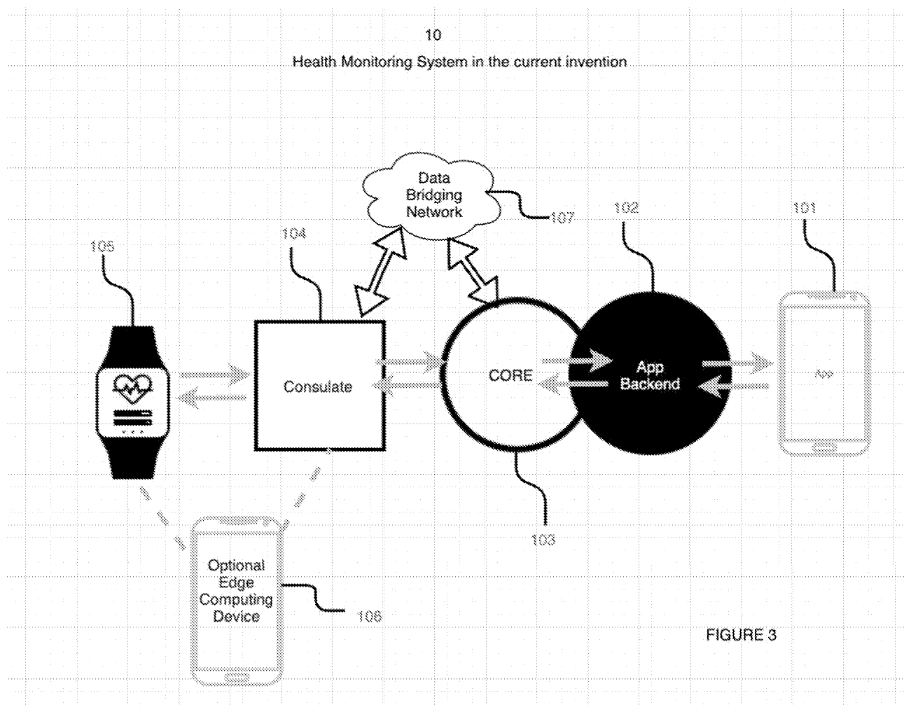

The physiological/vital data derived from the wearable 105 is sent via consulate 104 to the core 103 of the RHMS 10 to ultimately be displayed on a remote user interface/dashboard/application 101, as shown in FIG. 3. In an aspect, the remote user interface/dashboard/application 101 can take the form of any smart device, including, but not limited to, smart phones, tablets, lap-tops, and other general computing devices known in the art that include user interface tools and displays and that are configured to run various applications on their systems, as well as communicate wirelessly with other devices, utilizing various wireless networks, while also having access to the internet.

The data is sent with low-latency (i.e., short response time). The goal of the RHMS 10 is to have continuous and near real-time updates to a user's health data along with high-resolution patient histories. The dashboard 101 can display a dynamic patient stability score, generated from the health data, that monitors the health of the patient. The dashboard 101 also notifies the monitor of any anomalies or disturbances in the user's health. The RHMS 10 records the health data of any user whether they are healthy or ill and allows a monitor to have access to that information, via the remote user interface/dashboard 101, for various uses such as critical health decision making purposes, identifying the source of an illness, intervention prior to illness, health tracking, training, etc.

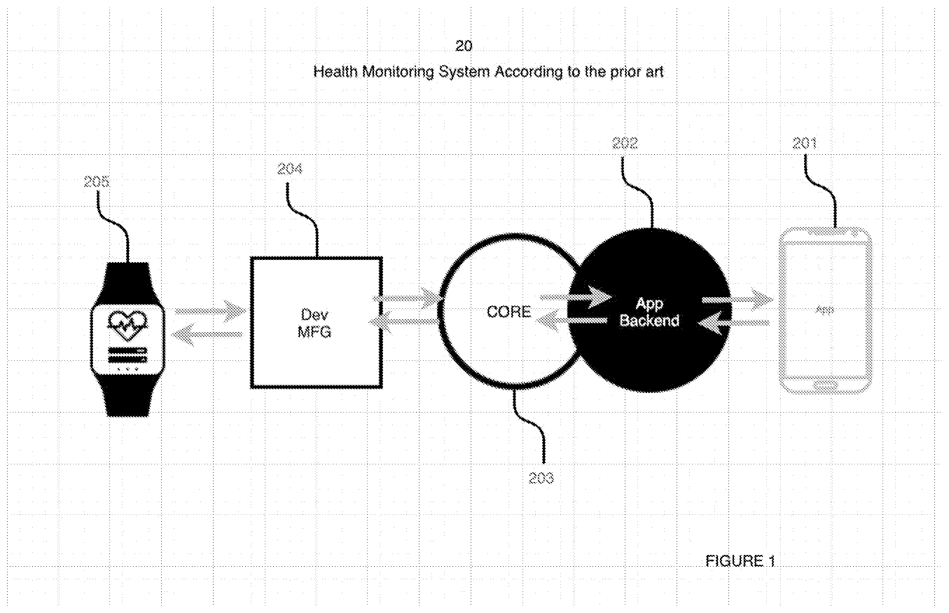

The reason that centralizing patient's health data, past and current, has been unsuccessful in the past is due to the fact that the infrastructure that is available traditionally requires an advanced edge computing device 201 to trigger the data collected (see FIG. 1). All the control for the interaction within systems of the prior art happens at the servers which hosts the core 203 and the cloud backend 202. In other words, the backend 202/core 203 controls the operation of the computing device 201, and has to activate the computing device 201 which hosts the application 201, which in turn reaches out to the wearable device 205. The time it takes to wake up the application 201 and trigger the necessary actions to retrieve data from the wearable 205 causes a delay and doesn't allow for real-time data collection, as opposed to triggering the backend 202/core 203 by the wearable device itself.

As shown in FIG. 3, the RHMS 10 includes a consulate 104. In an aspect, a consulate 104 is a standardized interface/software that is integrated in the cloud/server (core 102/application backend 103) that utilizes a wearable device 105. The consulate 104 allows communication between the wearable device 105, the server (core 102/app backend 103), the data bridge network (DBN) 107, and the dashboard/remote user interface 101. The wearable device(s) 105 can communicate bi-directionally with the consulate 104. In such aspects, the wearable 105 is capable of direct-to-cloud communication (for example, WiFi, LTE, LTEm, etc.), or otherwise, utilizing an optional edge computing device 106 connected to the server (core 102/app backend 103), to communicate with consulate 104. Additionally, the wearable device 105 can trigger data collection and can control/startup the RHMS 10 by initiating communication with the consulate 104. This allows for low latency/fast response time of the RHMS 10 and allows for a near real-time turnaround for data collection by reducing the amount of time required to send over the data. The wearable device 105 can send data as frequently as needed to the consulate 104, whether that be each predetermined time period or based on rules. These rules and time period can be integrated into the computing means on the wearable devices 105, or edge computing device 106. In addition, when there is data that is out of the norm or the user is experiencing a health issue, the wearable device 105 can, on its own, send information to the system 10 immediately. The RHMS 10 no longer has to ask the wearable device 105 for information, wait for a response, or have a device-cloud proxy.

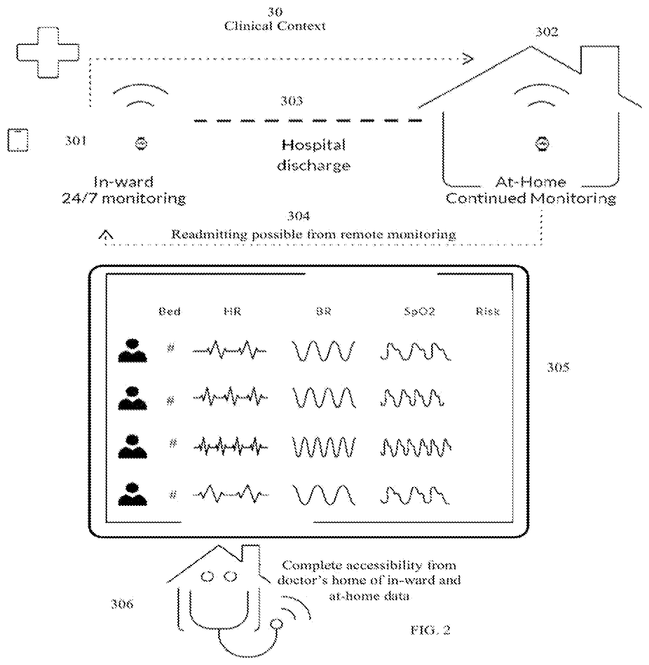

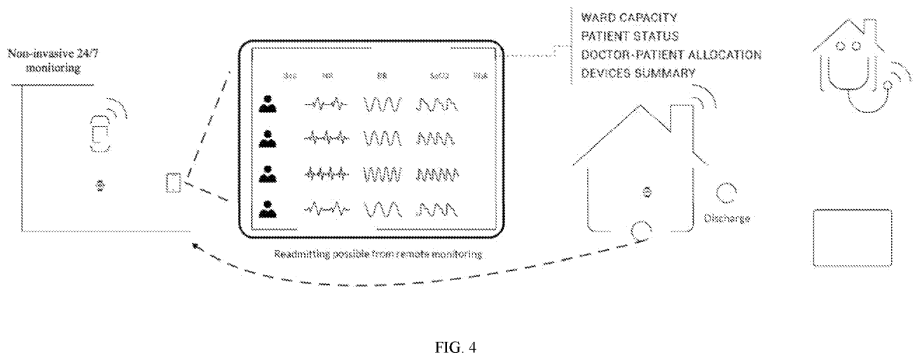

The RHMS 10 can be utilized in a clinical context 30, as shown in FIGS. 2 and 4-5. The RHMS 10, utilizing the dashboard 305, allows monitors/clinicians the ability to monitor a number of different patients via wearable devices 301 in a hospital setting (e.g., showing patients in a bed on the dashboard 305, as well as ward capacity, patient status, doctor-patient allocation, and device summaries (See FIG. 4)) that provides in-ward continuous and uninterrupted monitoring (i.e., 24/7). Also, those patients can continue to be monitored after being discharged (see 303) while at home 302, via the wearable device 301 as well. If a monitor/clinician, via the dashboard 305, sees that the health status of the discharged patient requires them to be readmitted (see 304), the system 10 can assist. The dashboard 305 allows a doctor access to the health data while in-ward or at-home 306.

### Health Monitoring System Data Analysis

Once the data of the user via the wearable device(s) 105 is collected, the data can be used to derive health data on the user and organize the data in a useful matter. In an aspect, the data can be de-identified (i.e., removing personal information that identifies the user) and cannot be tied to a specific user from within the system. In such aspects, the personal user information is only accessible by an authorized user through a separate application. The process is generalized so that any wearable device 105 may be utilized as long as the wearable device 105 may be configured to connect with the application/dashboard 101, as shown in FIG. 3. In an aspect, the wearable device 105 is enabled to utilize near field communication (NFC) in order to allow for the transfer of information from one wearable device 105 to another to ensure continuity when switching between wearable devices 105. The wearable device 105 may also use Bluetooth signal to locate the user as well as the user's proximity to other users/devices.

### De-Identification—

The data is separated from the personally identifiable information (PII) using a de-identification process. An example of this process is done using a Data Bridge Network (DBN) 107 (see FIG. 3) which works as an intermediary between the wearable device 105 and the cloud 103 and separates the data and creates unique identifiers for the user and for the application 101 accessing the information. If the system 10 is compromised or breached, the data is anonymous and cannot be tied back to a specific user.

The DBN 107 registers each new wearable device 105 as a new “data source” and gives the source a unique identification number. When a different party consumes this information, the data is transferred with a new unique identifier. The core 103 will not accept any information with PII, as it has no place to store such information. The DBN 107 simply facilitates the connection between the two parties and the data flow, it does not hold information itself. This process is further disclosed in U.S. Pat. No. 10,749,844 which herein is incorporated by reference in its entirety.

### New Generalized Process—

In an aspect, the RHMS 10 can use any wearable device 105 from any manufacturer configured to connect to the dashboard/application 101 (e.g., the LifeQ application). The wearable device 105 can be integrated with the consulate 104 with pre-packaged DBN 107 integrations. This allows the wearable device 105 to connect with the consulate 104 and communicate with the DBN 107 with a fast response time.

### Maintaining Continuity—

In order to ensure that there are no gaps in data intake in the RHMS 10, the wearable device 105 is enabled to use short range communication means (e.g., near-field communication (NFC) medium) that allows the devices 105 to be aware of nearby wearable devices 105 also enabled to communicate with other short range communication means. This is a short range communication method much like tap-to-pay on a mobile device. For example, when the battery is running low on one device 105 it will send an alert via consulate 104 to change devices 105. When a patient takes a fully charged wearable device 105 the two devices 105 (for example a low charged one and fully charged device) can recognize each other and transfer the user data and identification from one device to another. This creates a single data stream which allows for continuity in data intake and circumvents issues related to battery life without having to deal with overlaps.

Alternatively, the wearable devices 105 can utilize other means of wireless communication. For example, if there is a strong enough Bluetooth connection, the transfer of data can happen between the full charge device 105 and the low charge device 105. If neither is an option (NFC or Bluetooth), there is a manual process available to transfer devices much like current data transfer methods (WiFi, download, USB).

### Locating Users—

The wearable devices 105 leverages various wireless communication means (e.g., Bluetooth, WiFi, etc.) for location triangulation to keep track of the users and other devices. This is done by measuring the signal strength of the device 105 in relation to other device (wearables or other wireless access points). For example, in a hospital, this mechanism will allow monitors to know the location of the users of wearable devices 105, nurses that are wearing wearable devices 105, and medical devices that have wireless access points. In this use case, the doctors and nursing staff would be able to use this formation to locate lost patients, keep track of which nurses have come in contact with the patient, and which machines were near the patient. This data can further be analyzed to help in various situations such as contact-tracing or nurse scheduling.

### Monitoring System—

The wearable device collects user data and then utilizes the data to assist in monitoring a user's health in various contexts.

### Step 1: Collecting Data

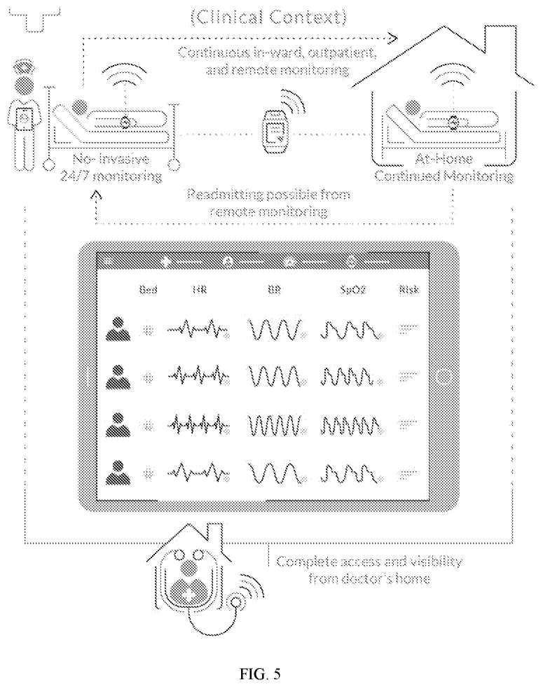

The basic objective of the RHMS 10 is to discover and monitor the physiological state of a user (e.g., a patient), via a wearable device 105, in various settings, including a hospital setting (see FIGS. 2 and 5), an out-patient/discharged patient (FIGS. 2 and 5), and normal home monitoring. The system includes at least two input classes: (1) Vital signs data (e.g. Heart Rate, Breathing Rate, SpO2) and (2) Demographic information (e.g. Age, Gender). The demographic information may be derived from previously collected wearable data or from additional sources such as values provided by the patient, information from a second device, or data from external sources such as medical health records. The data acquisition device 105 is configured to collect the patient's vital signs data. In an aspect, the wearable device 105 is configured to collect, determine, and transmit low-latency PPG-derived bio-signals to a dashboard 101. By utilizing a wireless network connected to the internet, the information can be communicated at high volumes with minimal delay. This allows for a near-real time update to user data. The demographic information may be supplied via the wearable device 105 as well, in instances in which the user may be prompted with questions to illicit such responses. Monitors may also collect and provide such information the RHMS 10 (e.g., via other computing devices 106 or dashboards 101). In other aspects, the demographic information may be provided via electronic medical records that are stored on the core 103/app backend 102 or other file storage means known in the art to which the RHMS 10 has access.

Beyond the vital signs, the RHMS system 10 can collect biological data, such as behavior and physiology, for example, details on sleep stages, efficiency, or amount of sleep, via a wearable device 105. This data can be used to trigger an alarm that will be informed by a wider set of information in addition to the vital sign information using the user's prior health history or any additional information provided to the application. Prior art monitoring systems are triggered when a certain vital sign threshold is surpassed such as a set heart/breathing/oxygen level. The RHMS 10 collects data that is complementary to a person's vital sign information that gives a monitor a broader picture of the user's health status. In an aspect, the data collected, and its analysis can be specialized to the individual users. This accounts for the purpose of monitoring (i.e., sports training) or pre-existing health data. Here, the RHMS 10 can have multiple data stream inputs either from multiple wearable devices or utilizing external sources of information such as the user's health history or an external medical device that allows monitors/clinicians to conclude more accurately than a method using a single data stream.

The data can be securely shared with a third-party interface (as described above) and in some instances may also be shared to additional devices. The input data is utilized to calculate a stability score. This data, along with the user's health history, is used to create a baseline health value for the user which is instrumental in accurately detecting anomalies in the user's health. The building of a user's baseline is described below.

Further, the data may include information such as geographic location and proximity to other individuals (e.g., users and physicians with devices 105 and/or dashboards 101), hospital equipment, and anything else configured to communicate with the device.

### Step 2: Analyzing the Data

### 2.1 Health Baseline and Anomaly Detection

The user's health baseline is calculated by building a model of an individual's physiological history and current health state. This baseline health value acts as another checkpoint before an anomaly alert is triggered. This is useful because there are instances when someone has a higher resting heart rate which would normally be flagged as an “irregular anomaly” when compared to clinical thresholds but is completely healthy for this particular user. Therefore, the system is configured so that over time the user's health baseline is updated and personalized to account for the normal activities and physiological behavior of each individual user. Through this process, anomaly detection is more accurate as it is limited to deviations from the individual's baseline health and not general thresholds. This process is further disclosed in International Patent Application No. PCT/US2021/029940, filed Apr. 29, 2021, which herein is incorporated by reference in its entirety.

The system can further make use of questionnaires in order to communicate with the user and better assess whether the information reflects a serious condition that requires attention or whether the signals are confounders which are not medically relevant. The questionnaires are presented on a digital display to the user either on the wearable device 105 or using an optional edge computing device 106. In some aspects, the questionnaires can be presented to monitors via the dashboard or similar devices so they can assist the patient/wearable device user 105 provide answers. The responses may contribute to the data analysis and accuracy of data collection. These questionnaires may also be triggered in instances where an illness/complication is confirmed. The individual would periodically be asked questions to see where their health is at and how their health/recovery is progressing. This also may be used to collect general information regarding the lifestyle of the particular individual to assess their health;/progress. An example of such a use could be in the case where Covid-19 has been detected in a user. That user's data would be collected to monitor the health of the user and the user would be asked questions about their symptoms, pain, etc. Further, additional information such as Global Positioning System (GPS) location (e.g., via the wearable device 105 or edge computing device 106) could be used to assist in contact-tracing or identifying the source of contract.

In an aspect, the RHMS 10 can determine the distribution of physiological parameters in isolation (e.g. what is the mean and standard deviation for a particular vital sign in the simplest case), or in combination where it is studied how different physiological parameters co-vary over time (e.g. what is the covariance between heart rate and breathing rate for an individual). Whenever new data is observed that seems unlikely to have originated from the personalized distribution described, an anomaly can be raised or the triaging priority for the patient can be increased. Anomaly detection may also take place in the cloud, which reduces latency and increases computational resources. As mentioned before, this takes into account that some deviations might be healthy even if they appear infrequently. For example, a decrease in heart rate to a lower, healthy rate for someone who generally has a high heart rate might be anomalous to this individual because they have not previously been observed to have a low heart rate, however, should not trigger an alert or increase triaging priority. Essentially, the baseline allows the RHMS 10, and the clinician/monitor, to consider only rare deviations towards an unhealthy state rather than report deviations to a healthy state. These models can be built using basic statistical concepts such as mean, standard, and covariance, but there are also much more sophisticated technologies that exist today such as Bayesian networks and deep learning models that provide more sophisticated technologies to capture, distribute, and calculate the likelihood that new data forms such a deviation.

### 2.2 Stability Score

The stability score utilizes the user's vital signs that are derived from the wearable 105 and compares them to the general clinical thresholds to provide an aggregate score to continuously determine the stability of the patient. In an aspect, the system utilizes a point-based system that is separated into categories, in which points are awarded based on a user's physiological data in relation to clinical thresholds. The median value of each physiological parameter during a time segment is compared against predetermined thresholds to determine the number points awarded to the patient. The stability score is calculated as the sum of the points from each category. The maximum score attainable in the sample model described below is 8, which would indicate maximum instability of the patient, advocating for said patient to receive more attention.

The following table shows the vital signs thresholds data and point allocation used to inform a sample model:

| Vital Sign | Threshold definition | Description | Points |
|---|---|---|---|
| Heart rate | 60 ≤ bpm ≤ 100 | Normal (5th | 0 |
| (HR)² | | percentile ≤ | |
| | | bpm ≤ 95th | |
| | | percentile) | |
| | bpm < 66 | Bradycardic (60 bpm) | 1 |
| | OR | OR | |
| | 90 < bpm < 133 | Tachycardic (100 bpm) | |
| | bpm ≥ 133 | (bpm ≥ mean + 4 | 2 |
| | | standard deviations) | |
| Breathing | 12 ≤ rpm ≤ 20³ | Normal breathing rate | 0 |
| rate (BR) | | range for an average | |
| | | adult; Eupnea | |
| | 8 < rpm < 12 ⁴ | Bradypnea | 1 |
| | 20 < rpm < 25 ³ | Tachypnea | |
| | rpm ≤ 8 ⁵ | Respiratory | 2 |
| | rpm ≥ 25 ⁶ | depression and | |
| | | tachypnea indicative | |
| | | of clinically relevant | |
| | | physiological or | |
| | | psychological activity | |
| SpO₂ ⁷ | SpO₂% ≥ 95 | Normal | 0 |
| | 90 ≤ SpO₂% < 95 | Hypoxic | 2 |
| | SpO₂ < 90% | Clinical Emergency | 4 |

The table above is one example of a stability score. Other aspects of the invention can utilize other physiological parameters to create a stability score that utilizes different point values, or other outputs.

### 2.3 Utilization of the Data

Anomaly detection using the user's baseline and stability score calculation can occur simultaneously. While the stability score utilizes predetermined physiological thresholds, the anomaly detection feature focuses on the individual's baseline health and any deviations from that baseline. Both allow for more accurate monitoring and have many applications.

One sample use of the RHMS 10 is remote hospital ward monitoring. In this situation, the stability score may be utilized as one of the options for patient triaging (i.e., priority ranking patients based on urgency of their conditions). Another method for patient triaging could be using the anomaly detection and deviation from the personal baseline. In each of these uses, the patients with higher deviations could be moved up on the priority list and hospital staff, for example, would be able to more effectively visit patients based on the urgency of their situation.

The user's information will be available remotely via a Patient Dashboard 101. The dashboard will allow the doctors, nurses, and authorized users to receive alerts when there are significant changes to the patient's information or when there is an update to triaging. The dashboard will also be accessible at any time in order to check in on the patient and make sure that the user is stable.

Further, the system can be configured to allow access of the information to the user via an edge computing device 106. This would allow the user to view their own health information as well as respond to questions prompted to them using a mobile device. The device may also contribute additional information such as GPS location, proximity measures, and other useful data.

### 2.4 Sample Applications of the System

### (a) Examples of Uses for Preventative Care

### (i) Example of the Use of Gathering Data on Healthy Subjects Prior to Entering the Care System (at the PCP Level):

Patient X has been monitored longitudinally for a year with a wearable device 105 capable of collecting low-latency PPG-derived bio-signals. Patient X's data can be monitored and viewed on a remotely accessible primary care physician (PCP) dashboard 101. When an anomaly is detected, the PCP is alerted about a gradual increase in ectopic beats in Patient X. The PCP refers X to a cardiologist. The RHMS 10, via the backend app 102/core 103, provides a report on changes to the ectopic beat frequency over the past year, as collected the wearable 105, directly and indirectly (i.e., talking to other wearables and devices associated with Patient X) to the cardiologist. The cardiologist is then able to use this information and may conduct additional, more invasive data collection (such as, but not limited to, collecting patient's ECG, blood pressure, echocardiogram) to determine that the patient is developing an increased risk of ventricular tachycardia. The patient is risk stratified accordingly and evaluated for treatment options such as getting an ICD (implantable cardiac defibrillator). The wearable device 105 remains in use by Patient X, and detects that the ectopic beat frequency has stabilized on the current treatment plan and is maintained.

This sample application can be applied in two scenarios. One where a diagnosis is made and a treatment is applied. Another is where there is not a diagnosis, but a prevention treatment plan is entered.

### (ii) Example Use of the Monitoring and Management Phase:

In this scenario, Patient X is in a similar situation, but has arrhythmias developing and has undergone cardioversion to reverse AFib. Patient X can then be monitored at home using the RHMS 10. Since ˜30% of cardioversion attempts fail in the long run and revert to AFib, outpatient monitoring provides notification to both the patient and specialist who will be made aware of any failures which allows for early, accurate detection and intervention.

As discussed above, an advantage of the RHMS 10 is that it is very commercially valuable when compared to what is currently available in other systems. The RHMS 10 can also change how hospitals operate and what is done within the walls and what can be done remotely, by the hospital, blurring the lines between in-patients and out-patients, creating continuity in health care and expanding hospital care beyond its physical walls. It would also help ensure that the patients that are physically at the hospital are those that really need to be there. This manages hospital space and equipment more intelligently.

(iii) Example of Detecting the Efficacy of Interventions on the Patient:

For many drugs, genetic variability affects whether patients respond well. The RHMS can use the physiological characteristics modelled on the user's physiological data, generated from long term use of the wearable device, to detect subtle changes in physiology indicative of effective action versus drugs that are not effective in a particular patient. A PCP or specialist can then assess this information to determine whether they should continue running the current script for the patient or update to a new drug that may be more efficacious.

### (b) Examples of Uses for Emergency Care

(i) Example of Patient Developing an Adverse Event after Heart Surgery in a General Ward:

In this case, Patient X would be equipped with a wearable device 105 post surgery which would alert the nursing staff (via the dashboard/application 101) of any adverse event allowing the staff to intervene and save the patient's life. Nurses visit general patients at an hourly to four hourly cadences at best, this technology would report vitals every fifteen minutes decreasing timeliness and potentially saving lives.

(ii) Example of Patient Developing an Adverse Event after Heart Surgery after being Discharged:

If the patient is monitored by the RHMS 10, including the wearable device 105, the dashboard/application 101 can alert at low latency a third party medical care provider of any anomalies. The care provider can then respond and check in on the patient to ensure that they receive the necessary care (especially in cases where the patient is not aware of the complication/loss of consciousness).

### 2.5 Additional Possible Uses for Contextual Health Data

The contextual health data of a user is useful because there are multiple things being monitored. This contextual medical picture is highly unique. What is detected includes a wide span of anomalies and then the physician has context of the data. Diagnosis and preventative treatment also would benefit from this additional information. You can also use the data to mine back from the healthy period until the patient is facing the physician. The backward context of the patient can point to a different system (ex. sleep) and detect other problems that need treatment. A cardiologist, for example, can come to more accurate and completely different conclusions with such information.

A good example of how this data can be utilized is in determining a user's Covid-19 infection risk. This process is further disclosed in International Patent Application No. PCT/US2021/029940 which herein is incorporated by reference in its entirety. This takes into account the user's vital information but also benefits from knowing the user's lifestyle, health, fitness, eating habits, geographic location, etc. The prognosis is more accurate because the data not only allows one to see the trajectory that has led to that state but also the future inferred causal trajectory.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

What is claimed is:

1. A remote health monitoring system for non-invasive health monitoring comprising:
a. a non-invasive instrument configured to acquire at least vital sign data of a user and to communicate wirelessly; and
b. a core configured to communicate wirelessly with the non-invasive instrument, wherein the non-invasive instrument and the core include at least one processor, and wherein either the non-invasive instrument or the core is configured to:
i. process the at least vital signal data to generate biological metrics;
ii. process the biological metrics to reflect a real-time user health state reflecting physiological data and behavioral data of the user; and
iii. detect deviations in the physiological data and the behavioral data of the user from the real-time user health-state.

2. The system of claim 1, further comprising at least one additional device that captures medically relevant data of the user, wherein the at least one additional device is further configured to communicate wirelessly with the non-invasive instrument, and wherein the core or the non-invasive instrument is configured to process the medically relevant data from the at least one additional device with the biological metrics to reflect the real-time health state of the user.

3. The system of claim 1, wherein the real-time user health-state is reflected by generating a patient stability score, wherein the patient stability score is calculated by awarding points based on the physiological data of the user in relation to clinical thresholds, wherein the patient stability score is utilized for low-latency patient triaging.

4. The system of claim 3, further comprising a remote user interface configured to be in wireless communication with the core or the non-invasive instrument, wherein the biological metrics, the real-time user health state, the physiological data, the behavioral data, and all other information related to the health of the user, is accessible on the remote user interface.

5. The system of claim 4, wherein the core and the non-invasive instrument are configured to send alert messages to the remote user interface based upon the physiological data of the user in relation to the clinical thresholds.

6. The system of claim 4, wherein the system has access to electronic heath records of the user, wherein the electronic health records, the patient stability score, and the detected deviations are used in combination to assess a physiological status of the user.

7. The system of claim 1, wherein the system is configured to generate a baseline physiological state of the user from the biological metrics collected over a period of time, wherein the detection of deviations takes into consideration the baseline physiological state of the user.

8. The system of claim 1, wherein the non-invasive instrument comprises a plurality of non-invasive wearable devices, wherein each of the plurality of non-invasive wearable devices is further configured to:
a. communicate with each other determine which one of the plurality of non-invasive wearable devices should collect data while more than one of the plurality of non-invasive wearable devices is in use;
b. simultaneously collect data from two or more devices during a transition period to maintain data continuity;
c. communicate with each other to determine which should be using battery power and which should be preserving battery power at a given point; and
d. share data with each other to ensure continuity in battery life as well as data collection.

9. The system of claim 8, wherein the plurality of non-invasive wearable devices is configured to continuously monitor the user to collect general health lifestyle information to generate healthy physiological state and health status of the user.

10. The system of claim 9, wherein the collected general health lifestyle information of the user is collected during a healthy period and is used to identify a cause of a new medical condition arising during a later period for the user.

11. The system of claim 10, wherein the general health lifestyle information of the user is collected during a healthy period and is used to predict disease course in the instance of a new disease arising.

12. The system of claim 11, wherein an intervention plan is generated based on the prognosis derived from the general health lifestyle information of the user plus measured behavior of the user.

13. The system of claim 1, wherein the core is configured to send the real-time user health state and the detected deviations of the user to one or more authorized persons, wherein the authorized person includes a family member, a caretaker, or a medical health professional.

14. The system of claim 1, wherein the real-time user health state and the detected deviations of the user may be sent to one or more authorized group, wherein the authorized group is a pharmaceutical company, a health insurance company, or a medical company.

15. The system of claim 1, wherein the non-invasive instrument is configured to detect proximity data of at least one or more non-invasive instruments using near field communication.

16. The system of claim 15, wherein the proximity data is utilized for prediction and allocation of clinical staff and resources.

17. The system of claim 15, wherein the proximity data is utilized to determine contact amount between users of the one or more non-invasive instruments and clinical staff.

18. The system of claim 15, wherein the non-invasive instrument is further configured to detect proximity of at least one or more medical devices.

19. The system of claim 1, wherein the non-invasive instrument is a wearable device that includes at least one photoplethysmography (PPG) sensor to obtain the at least vital sign data of the user.
